(19) United States
(12) Patent Application Publication
(10) Pub. No.: US 2005/0234430 A1
(43) Pub. Date: Oct. 20, 2005

(54) Method and apparatus for patient-controlled medication delivery

(76) Inventors: Jianren Mao, Arlington, MA (US); Shihab U. Ahmed, Lincoln, MA (US)

Correspondence Address: DALY, CROWLEY, MOFFORD & DURKEE, LLP, SUITE 301A, 354A TURNPIKE STREET, CANTON, MA, 02021-2714, US

(21) Appl. No.: 10/825,069

(22) Filed: Apr. 15, 2004

(51) Int. Cl. A61K 9/22

(52) U.S. Cl. 604/890.1; 128/DIG. 13

(57) ABSTRACT

A method and apparatus for providing patient-controlled medication delivery is presented. The apparatus includes a medication storage subsystem, an interval timer, a patient verification system coupled to the medication storage subsystem, and a delivery subsystem which delivers one of the prescribed amounts of medication if the interval timer has reached an end of a predetermined time interval and the patient verification system verifies an identity of the patient. The method for providing patient-controlled medication delivery includes verifying an identification of an intended patient and providing a first prescribed amount of a medication for the intended patient, wherein the intended patient determines a time to start taking the prescribed amount of the medication. The method further includes waiting a predetermined time interval, again verifying the identification of the intended patient and providing another prescribed amount of the medication for the intended patient.

### CROSS REFERENCE TO RELATED APPLICATIONS

Not Applicable.

### STATEMENT REGARDING FEDERALLY SPONSORED RESEARCH

Not Applicable

### FIELD OF THE INVENTION

The present invention relates generally to a medication delivery system and more particularly to a patient-controlled medication delivery system.

### BACKGROUND OF THE INVENTION

Many people must take prescribed medication over a period of time. In order for the prescribed medication to be most effective, patients are typically required to take the prescribed medication on a defined schedule (e.g., every 12 hours). A problem associated with taking prescription medications on a defined schedule is non-compliance of patients with respect to the taking of the medication at the appropriate time. Non-compliance with the prescribed medication schedule can lead to several problems. One problem is that such non-compliance can result in the patient suffering from effects the prescribed medication is intended to control (e.g., high blood pressure). Another problem is that patient non-compliance makes it difficult for a doctor or other medical practioner to determine whether the medication is working properly or if the dosage amount or time interval between dosages needs to be changed to provide more effective relief.

When a patient is prescribed with a controlled substance, such as a narcotic pain killer, non-compliance with the prescribed medication may lead to serious and even fatal consequences.

In addition, tampering with the prescribed medication could cause serious legal and medical problems. There are several reasons for unintentional non-compliance with a prescribed medication dosage schedule. Patients, especially the elderly or mentally ill, may not remember the time specified or the proper dosage for taking a medication. Further, patients may forget if they have already taken a dosage and inadvertently take another dosage or patients may skip a dosage, thinking they had already taken the dosage. This may result in the patient either not taking the medication or taking too much of the medication, possibly leading to an overdose.

Another reason for unintentional non-compliance with a medication dosage schedule is people may have schedules that vary every day, and thus they are not available to take medication at the same time every day. The patient may be working, traveling or otherwise unable to follow the schedule for taking their prescribed medication.

Another problem associated with patient non-compliance with a schedule for taking medication is patient identification regarding the prescription. In a situation wherein multiple people are co-residing, it may be easy for the medications to get confused, with the result that a patient takes a medication intended for another person, which can lead to problems.

### SUMMARY OF THE INVENTION

In accordance with the present invention, a method and apparatus for providing patient-controlled medication delivery is presented. The apparatus includes a medication storage subsystem for storing prescribed amounts of a medication, an interval timer coupled to the medication storage subsystem, a patient verification system coupled to the medication storage subsystem, and a delivery subsystem which delivers one of the prescribed amounts of medication when the interval timer has reached an end of a predetermined time interval, when the patient verification system verifies an identity of the patient, and when the patient activates the medication delivery system. The patient selects the delivery time of the first dosage and therefore determines the best time to receive the dosages.

A method for providing patient-controlled medication delivery includes verifying an identification of an intended patient and providing a first prescribed amount of a medication for the intended patient, wherein the intended patient determines a time to start taking the prescribed amount of the medication. The method further includes waiting a predetermined time interval, the time interval starting from the time the intended patient last requested and received a prescribed amount of the medication, again verifying the identification of the intended patient and providing another prescribed amount of the medication for the intended patient.

### BRIEF DESCRIPTION OF THE DRAWINGS

The foregoing features of this invention, as well as the invention itself, may be more fully understood from the following description of the drawings in which:

FIG. 1 is a perspective view of an apparatus for providing patient-controlled delivery of a medication;

FIG. 2 is a cross-sectional side view of an apparatus for providing patient-controlled delivery of a medication;

FIG. 3 is a block diagram of a medication storage subsystem of the patient-controlled medication delivery system;

FIG. 4 is a diagram of a piston of a medication delivery subsystem; and

FIG. 5 is a flowchart of a method of providing patient-controlled medication delivery.

### DETAILED DESCRIPTION OF THE INVENTION

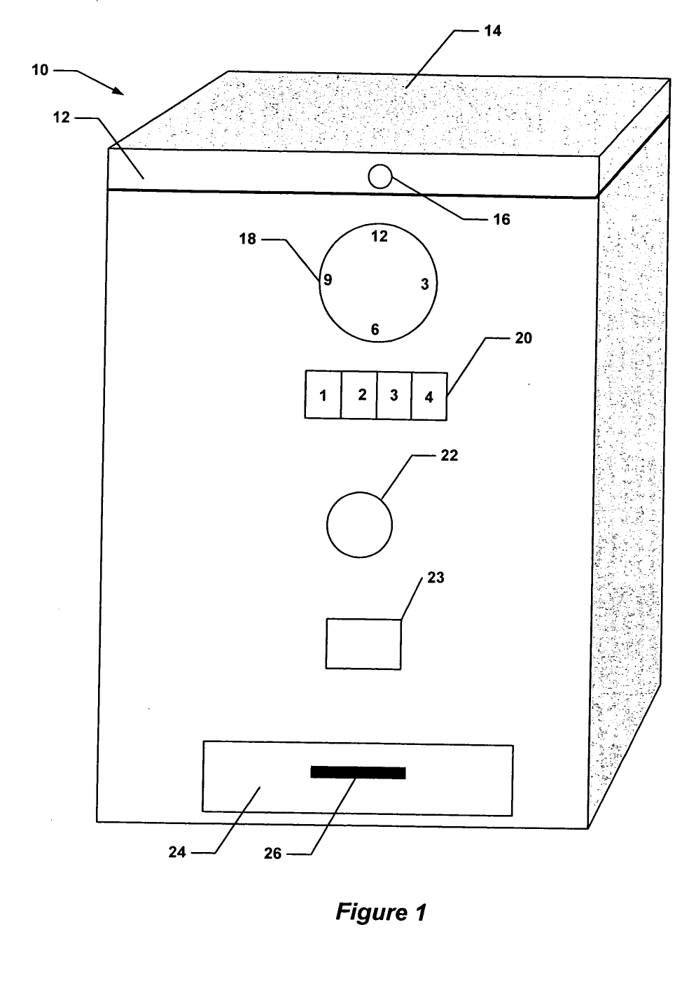

Referring now to FIG. 1, an apparatus 10 for providing patient-controlled delivery of a medication includes a container 12 having a movable access panel or cover 14 coupled thereto. The access panel 14 may be secured to the container by a locking mechanism 16 (or more simply a “lock” 16) which prevents an unauthorized user from accessing the inside of the container 12. In the exemplary embodiment shown in FIG. 1, the access panel 14 is shown as a top cover 14 to the container 12. Those of ordinary skill in the art will appreciate of course that access panel 14 can also be provided as a bottom cover or side cover or as any removable portion or portions of container 12.

The apparatus 10 includes a timer 18, a patient identification verification system 20, an activation switch 22, a drawer 24 having a handle 26, and an indicator 23. Not visible in this view are internal subsystems such as a medication storage subsystem and a medication delivery subsystem.

The container 12, in a preferred embodiment, is tamper proof and made of a relatively strong material such as stainless steel or the like. Container 12 has located therein a medication storage subsystem and a medication delivery subsystem which are used in combination to provide medication at predetermined time intervals to a properly identified patient. The patient selects the delivery time of the first dosage by completing a patient identification process once the time interval has elapsed. Thus, the apparatus 10 functions such that the patient has the freedom to determine the time to begin taking the medication.

The lock 16 secures cover 14 in a closed position to thus prevent unauthorized access into the container 12. In one embodiment, the lock is operable by way of a key (not shown). It should be appreciated, however, that other types of lock systems (e.g., a keyless lock system) could also be used. The cover 14 can be opened or removed by an authorized person (e.g., a doctor, nurse or other medical practitioner) for the purpose of replacing the supply of medication, but otherwise is kept locked. This prevents patients or others from removing the medication supply or otherwise tampering with the contents of the container 12.

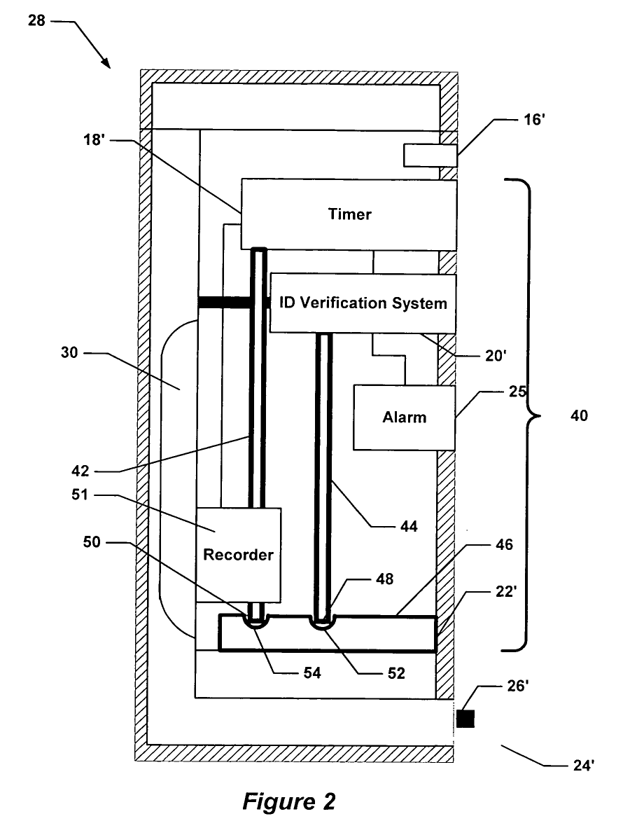

The timer 18 is used to measure the time interval between medication deliveries. In a preferred embodiment, the predetermined time interval between medication deliveries measured by the timer 18 is set by an authorized person such as a physician or nurse (according to physician instruction). The timer 18 is connected to a medication storage subsystem 30 (described below in conjunction with FIG. 2) and the medication delivery subsystem 40 (also described below in conjunction with FIG. 2). In the embodiment shown in FIG. 1, the timer 18 is mounted on a front surface of the container 12. It should, however, be appreciated that the timer may be located on other locations of the apparatus 10. It should also be appreciated that the timer may be physically separate from the apparatus 10 as long as the timer is coupled (e.g. by a wire or wireless connection) to the medication storage and delivery subsystems 30, 40.

The timer 18 controls the medication delivery subsystem 40 such that medication is only available after the timer has elapsed as indicated by the front indicator 23. Indicator 23 may be an audio indicator, a visual indicator, a combination audio and visual indicator or other type of indicator as would be known by one of ordinary skill in the art. Alternately, the indicator could be included as part of activation switch 22. Thus, once the time interval has elapsed, if the patient desires to have the medication the patient begins a medication dispensing process by performing a patient verification process (e.g. by pushing an appropriate sequence of ID keys).

Once the verification process is completed correctly, the medication delivery subsystem 40 can be activated. In one embodiment, the activation switch 22 is engaged once the verification process is correctly completed and a patient or other person (e.g. a caregiver) can operate the activation switch 22 (e.g. by pushing the activation switch) to get a single dosage of the prescribed medication. Medication delivery is not available if the time interval has not elapsed. This prevents a patient from taking too much medication during any one time period. After receiving the single dosage, the timer restarts and the patient is prevented from acquiring another dose of medication until the time interval elapses.

The patient-controlled medication delivery system also includes the patient identification verification system 20 for verifying the identity of the patient or verifying that the patient or other person has the requisite information to operate the system. The system 20 thus prevents unauthorized use of the apparatus 10.

In a preferred embodiment, the patient identification device is provided as a Personal Identification Number (PIN) device 20, although other identification verification systems could also be used (e.g. a biometric verification system could be used). The system 20 is in communication with the medication delivery subsystem 40 such that once a correct PIN is entered, the patient is able to have the medication delivered if the time interval between medication deliveries has passed.

In one embodiment the system 20 includes a digital touch-tone number pad. To operate the system, an ID number is entered which must match a predetermined patient ID number. Once the correct ID number is entered the patient can receive medication from the system.

In one particular embodiment, a four-digit ID system is installed. In this embodiment, a patient is allowed a predetermined number of attempts (e.g. three attempts) to enter a combination of digits corresponding to a valid ID number (the digits can be numbers, letters or a combination of letters and numbers). Three attempts are allowed considering the following factors: 1) a four-digit ID system will have 16 combinations and is not easy to break with three tries and 2) in elderly groups, a mistake could be made more than once when entering the ID by pushing the keys. However, after a predetermined number of attempts (e.g. three attempts) if the correct code has not been entered the system will be locked until the time interval between medication deliveries has elapsed.

Under such circumstances, the patient will not be able to receive medication from the apparatus 10 until the time interval has expired (as measured form the last of the three attempts). If the patient cannot wait that long to receive the medication, the patient will need to acquire the medication from another source, (e.g. by contacting or visiting the appropriate doctor or other medical practitioner or appropriate pharmacist). The patient will also have to contact the pharmacy or medical service provider (with appropriate identification) to have someone provide the patient's correct PIN number to them. It should be appreciated, of course, that ID's having fewer or more than four digits may also be used.

The apparatus 10 also includes the activation switch 22 which is in communication with the medication delivery subsystem such that, if the timer interval has elapsed and the patient identification has been verified, engaging the activation switch 22 activates the medication delivery subsystem and a prescribed dosage of medication is made available to the patient. In a preferred embodiment the medication is deposited in the drawer 24 that the patient can access to remove the dosage of medication. In one particular embodiment, the switch 22 is provided as a pushbutton switch 22 disposed on the front surface of container 22. It should be appreciated, however, that the activation switch 22 may be coupled to the apparatus 10 in any manner now or later known to one of ordinary skill in the art including via a wireless or hardwired connection.

In some embodiments there may be an adjustable predetermined time delay (e.g. a 10 to 30 second delay time) after the first push of the delivery activation switch 22 before the timer 18 locks the medication delivery subsystem 40 for the next set time interval. The time delay allows the patient to engage the activation switch 22 one or more additional times such that one or more additional dosages of a desired medication is dispensed. In the case where activation switch 22 is provided as a pushbutton switch, the delay allows the patient time to push the pushbutton switch again to have an additional medication dosage if so desired (according to the physician's recommendation). In most cases, one or two tablets or capsules are commonly used. However, in some versions of this device more than two pushes may be allowed to accommodate the prescription needs.

Activation switch 22 may include an indicator 23 (e.g., a light) visible to the user (e.g. from the surface of the activation switch), which is connected with the timer. In the case where the indicator 23 is provided as an indicator light, the indicator light could be seen, for example, as a first color or light pattern (e.g., red or flashing) during the locked interval and as a second color or pattern (e.g. green or solid) after the set time interval has elapsed. There may also be an alarm (e.g. an audio or vibratory alarm signal) system (designated 25 in FIG. 2) connected to the timer to remind the patient that the set time interval has elapsed and the device is ready to deliver the medication again if needed.

Referring now to FIG. 2, a cross-sectional side view of a patient-controlled medication delivery system 28 which may be similar to the system 10 shown in FIG. 1 includes a medication storage subsystem 30 (described below) coupled to a medication delivery subsystem 40.

The medication delivery subsystem 40 includes an activation timer 18′ which may be similar to the activation timer 18 described above in FIG. 1, an ID verification system 20′ which may be similar to the verification system 20 described above in FIG. 1, connecting rods 42 and 44, a piston assembly 46 and an activation switch 22′ which may be similar to the activation switch 22 described above in FIG. 1.

When either of rods 42 or 44 is deployed such that the distal end of the rod (50 and 48 respectively) engages the appropriate detent (52 and 54) in piston assembly 46, the piston disposed within piston assembly 46 is prevented from moving toward the medication storage subsystem 30, and the delivery of medication is prevented. On the other hand, when the time interval has elapsed timer 18′ will disengage rod 42 from piston assembly 46. Similarly, when a patient ID has been verified, rod 44 will also disengage from piston assembly 46. It is only when both rods 42 and 44 have disengaged the piston assembly 46 that piston may be activated by operation of activation switch 22′ to release a dosage of medication from medication storage subsystem 30. While a particular embodiment of the locking mechanism for the piston has been described, it should be appreciated that other locking means could be used, such as an electrically activated lock or the like.

Also shown in this Figure is a recorder 51, which is part of the medication storage subsystem 30. In one embodiment, the recorder 51 imprints the time on a sticker when the patient presses the activation switch 22′. This sticker may be removed and transferred to the patient chart to give the physician the exact timing and intervals of the medication uses. This will help physician to further adjust the medication accordingly such as increasing the dosage, decreasing the dosage or changing the time interval between dosages.

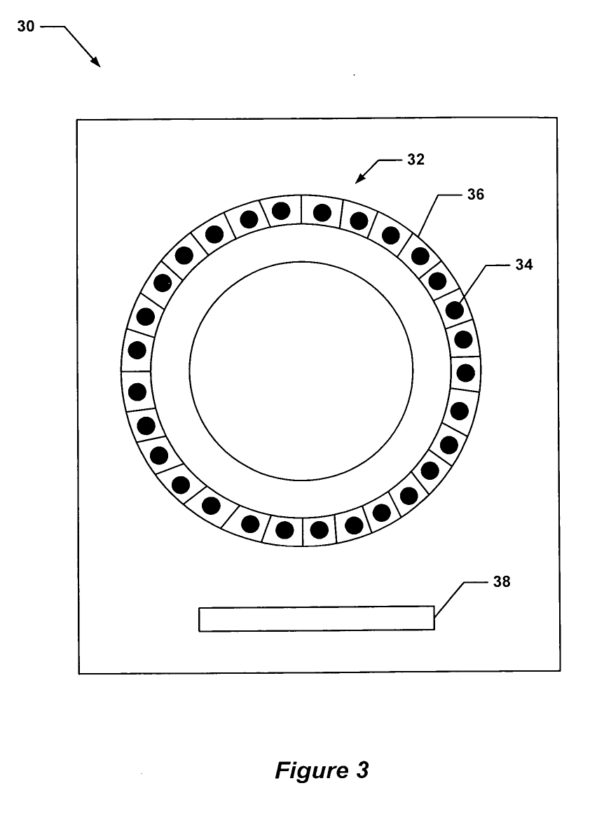

Referring now to FIG. 3, a medication storage subsystem 31, which may be similar to the medication storage subsystem 3o described above in FIG. 2, includes a medication container 32 in which medications 34 (e.g. tablet, capsules, pills etc.) are disposed. In the exemplary embodiment of FIG. 3, the medications 34 are in a medication package 36 have a disk shape (i.e. circular shape). As shown in FIG. 3, the medication 34 is disposed around a perimeter of the package 36. It should be appreciated that although the package 36 and container 32 are here shown having a round shape, those of ordinary skill in the art will appreciate of course that other shapes may also be used. Those of skill in the art should also appreciate that the medication 34 may be arranged in a pattern and location other than a circular pattern along a perimeter of a medication container 32 or package 36.

An authorized person (e.g., a medical practitioner such as a physician a nurse or a pharmacist) mounts the medication package 36 into the storage subsystem 30. At the appropriate time, the storage subsystem 30 dispenses an appropriate dose of the medication 34.

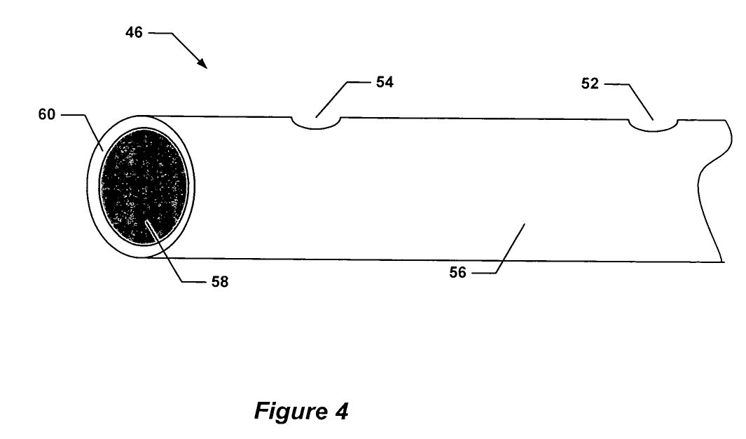

Within the subsystem 30 the circular medication package 36 rotates in a clock-wise direction each time a medication 34 (e.g. a pill) is pushed out from the package 36. When the patient pushes the activation switch 22 a hollow cylindrical shaped part of the piston (described in detail below in conjunction with FIG. 4) cuts the portion of the medication package 36 which serves the medication 34 (e.g. a pill) in the package 36. In some embodiments, the medication 34 is secured via a plastic or foil cover. A solid portion of the piston then pushes the medication 34 out of the medication storage subsystem 100 and into the delivery drawer. Also shown is a sticker 38. The sticker is used by the recorder (described above) to record the time the medication was delivered.

Referring now to FIG. 4 in conjunction with FIG. 2, the medication delivery system comprises a drawer 24 or similar structure for the purpose of receiving the pill after the pill has been dispensed (e.g., by having been pushed by the piston), making it available for the patient to pick-up. This drawer 24 is located at the lower end of the device 10 and extends across the bottom of the device. A patient has to pull the drawer 24 by a handle 26 located on the surface of the draw and is able to pick-up the pill.

An example of a preferred embodiment of the piston assembly 46 is shown in detail in although it should be appreciated that other types of piston assemblies could be utilized. In this example embodiment piston 56 is a cylindrically shaped structure with two indentations or detents 52 and 54 for the locking mechanism. Detent 52 receives the distal end of rod 48 which extends between the piston assembly 46 and the ID verification system 20. Detent 54 receives the distal end of rod 42 which extends between the piston assembly 46 and the timer 18′ (FIG. 2).

The piston assembly 46 comprises a cylindrical bar 58 disposed within a hollow structure 56 here shown having a tubular shape for example. The bar 58 may be solid in some embodiments. The tubular structure 56 is provided having a sharp circular edge 60 at a distal end. This tubular structure 56 is movable over a short distance and allows the solid cylinder 58 to slide forward within the tubular structure 56. The sharp edge 60 of the tubular structure is used to make a cut (in this case circular cut) around the plastic or foil containing the pill in the medication delivery subsystem 30. The solid bar 58 then pushes the pill out of the foil.

After the piston assembly 46 is activated by a patient through the activation switch (e.g. activation switch 22′ in FIG. 2) and delivers the medication, the connector rods 42 and 44 will lock, and prevent any forward movement of the piston assembly 46 for the set time interval and thus no medication will be delivered during the set time interval. Once the set time interval has elapsed, the connector rod 42 will release from piston assembly 46. If the patient desires to have the medication after the set time interval has elapsed, he or she is able to activate the piston assembly 46 by pushing the activation switch 22′ to get the medication, assuming the patient identification has also been verified and rod 44 has released piston assembly 46.

While a particular embodiment has been described, it should be appreciated that other embodiments are also within the scope of the present invention. For example, the timer 18 and the connecting rod 42 is connected together via a spring (may also be a magnetic system) mechanism. When the prescribed time elapses, the connecting rod 42 will un-lock itself from the detent 54 and remain in that position until the patient pushes the activation switch (e.g. activation switch 22′) to receive the medication.

The activation switch pushed by the patient to receive the medication, also has several other functions, including 1) rotating the cartridge containing the pills, 2) recoding the time of medication delivery on the sticker 51, 3) resetting the timer 18, and 4) locking the system (e.g. by placing the connecting rod 42 into the dent 54).

The delivery system could also be similar to a sliding door allowing pill(s) to slide out of the pill-cartridge, which may in a vertical fashion under this design.

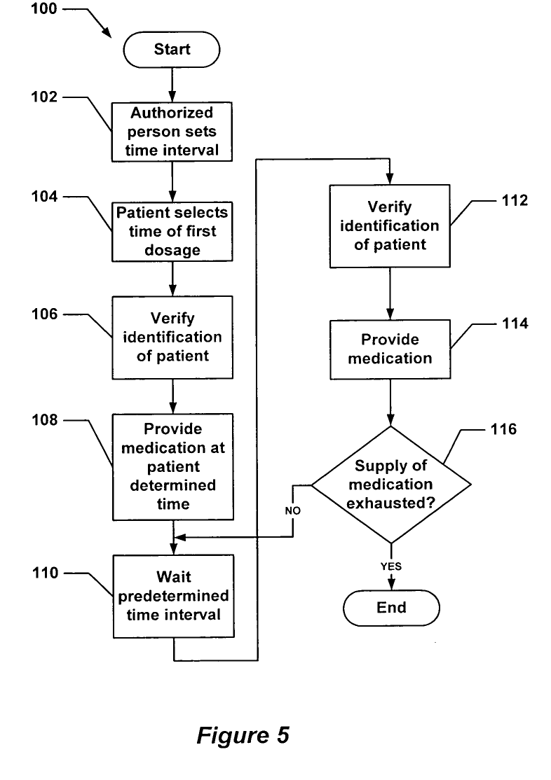

A flow chart of the presently disclosed method is depicted in FIG. 5. The rectangular elements are herein denoted “processing blocks” and the diamond shaped elements are herein denoted “decision blocks”. It will be appreciated by those of ordinary skill in the art that unless otherwise indicated herein, the particular sequence of processing blocks and/or decision blocks described is illustrative only and can be varied without departing from the spirit of the invention. Thus, unless otherwise stated the processing blocks and/or decision blocks described below are unordered meaning that, when possible, the processing blocks and/or decision blocks can be performed in any convenient or desirable order.

Referring now to FIG. 5, the process for providing patient-controlled medication delivery 100 starts and processing block 102 is executed. In processing block 102 an authorized person sets the time interval. The authorized person may be a physician or nurse (according to physician instruction). The time interval defines the time between medication deliveries.

In processing block 104 the patient (or other authorized person) selects the time of the first dosage. This is done at a time determined by the patient, not at a predetermined time of day. Thus, the patient has the freedom to determine the time to begin taking the medication.

In processing block 106 the identity of the patient is verified. In one embodiment, the patient is required to enter a patient ID number via a PIN device. Alternately, other patient verification means could be used, such as scanning a bar code from a patient wrist band, or a medication card having a bar code or magnetic strip thereon or a biometric verification system can be used.

In processing block 108 the medication is provided to the patient at the patient-determined time of day. One feature of the present invention is that the patient has determined the time of day to start taking the medication.

In processing block 110 the time interval between dosages of the medication is observed. This time interval is determined by a doctor or other authorized medical practitioner. While the patient determines the starting time to begin taking the medication, the doctor (or other authorized medical practitioner) determines the time interval between dosages of the medication.

Once the time interval has elapsed, in processing block 112 the patient identification is again verified. This done to assure that the proper patient will be receiving the medication. This step is preferably done in a similar manner as the verification of the patient that was conducted in processing block 106 described above.

In processing block 114 the medication is delivered. The patient has the medication made available to him or her at the predetermined time interval and after the identification of the patient has been verified.

In decision block 116 a determination is made whether the supply of medication has been exhausted. If the supply of medication has been exhausted, then the process ends otherwise blocks 110 et seq. are executed.

Having described preferred embodiments of the invention it will now become apparent to those of ordinary skill in the art that other embodiments incorporating these concepts may be used. Accordingly, it is submitted that the invention should not be limited to the described embodiments but rather should be limited only by the spirit and scope of the appended claims.

1. A patient controlled medication delivery apparatus comprising:
an activation switch;
a medication storage subsystem for storing a plurality of prescribed amounts of medication;
an interval timer coupled to said medication storage subsystem;
a patient identification and verification system coupled to said medication storage subsystem; and
a medication delivery subsystem wherein in response to said interval timer reaching an end of a predetermined time interval, said patient verification system verifying an identity of a patient and said activation switch being engaged said medication delivery subsystem delivers a prescribed amount of said medication.

2. The apparatus of claim 1 further comprising a recorder coupled to said medication storage subsystem, said recorder recording a time when said prescribed amount of medication was delivered to said patient.

3. The apparatus of claim 1 wherein said patient verification system comprises a personal identification number (PIN) system.

4. The apparatus of claim 1 further comprising a timer locking mechanism coupled between said interval timer and said medication delivery subsystem, said timer locking mechanism locking said medication delivery subsystem when said interval timer has not elapsed, and unlocking said medication delivery subsystem when said interval timer has elapsed.

5. The apparatus of claim 1 further comprising a patient verification locking mechanism coupled between said patient verification subsystem and said medication delivery subsystem, said patient verification locking mechanism locking said medication delivery subsystem when said patient verification system has not verified the identification of a patient, and unlocking the lock on said medication delivery subsystem when said patient verification system has verified the identification of a patient.

6. The apparatus of claim 1 wherein said activation switch includes at least one indicator light.

7. The apparatus of claim 1 wherein said at least one indicator light of said activation switch lights in at least one of a first color when said timer has elapsed and a second color when said timer has not elapsed.

8. The apparatus of claim 1 further comprising an alarm, said alarm coupled to said interval timer and wherein said alarm provides an indication when said interval timer has elapsed.

9. The apparatus of claim 1 wherein said medication delivery subsystem includes a piston assembly coupled between said activation switch and said medication storage subsystem.

10. The apparatus of claim 9 wherein said piston assembly comprises:
a hollow piston having a cutting edge at a distal end thereof for engaging said medication storage subsystem; and
a solid piston movably disposed within said hollow piston for engaging said medication storage subsystem.

11. The apparatus of claim 9 wherein said hollow piston includes a first detent for receiving a portion of a timer locking mechanism therein.

12. The apparatus of claim 11 wherein said hollow piston includes a second detent for receiving a portion of a patient identification locking mechanism therein.

13. A method of patient-controlled medication delivery comprising:
(a) verifying an identification of a patient for whom a prescribed medication is intended;
(b) providing a first prescribed amount of a medication for the patient, wherein said patient determines a time to start taking said prescribed amount of the medication;
(c) waiting a predetermined time interval, said time interval starting from the time the intended patient last took a prescribed amount of said medication;
(d) verifying the identification of the intended patient; and
(e) providing another prescribed amount of said medication for the intended patient.

14. The method of claim 13 wherein said steps c, d, and e are repeated until the supply of medication is exhausted.

15. The method of claim 13 wherein said providing a first prescribed amount of a medication further comprises recording information relating to a time said prescribed amount of medication was provided.

16. The method of claim 13 wherein said providing another prescribed amount of a medication further comprises recording information relating to the time said prescribed amount of medication was provided.

17. The method of claim 13 wherein said verifying the identification of an intended patient is done by way of a personal identification number (PIN) system.

18. The method of claim 13 wherein said medication comprises a medica-tion taken orally.
